(19) United States
(12) Patent Application Publication
(10) Pub. No.: US 2014/0356891 A1
(43) Pub. Date: Dec. 4, 2014

(54) Determining the Dynamic State of Analytes by Magnetic Flow Measurement

(71) Applicants: Oliver Hayden, Herzogenaurach (DE); Michael Johannes Helou, Regensburg (DE); Mathias Reisbeck, Obertraubling (DE); Sandro Francesco Tedde, Erlangen (DE)

(72) Inventors: Oliver Hayden, Herzogenaurach (DE); Michael Johannes Helou, Regensburg (DE); Mathias Reisbeck, Obertraubling (DE); Sandro Francesco Tedde, Erlangen (DE)

(21) Appl. No.: 14/238,943

(22) PCT Filed: Aug. 1, 2012
(86) PCT No.: PCT/EP2012/064991

§ 371 Date: Jun. 2, 2014

(30) Foreign Application Priority Data
Aug. 15, 2011 (DE) DE 102011080945.7

(51) Int. Cl. G01N 15/10; G01N 33/80; G01N 33/58

(52) U.S. Cl. CPC G01N 15/1031; G01N 33/585; G01N 33/80; G01N 2015/0065
USPC 435/29

(57) ABSTRACT

Devices and methods for magnetic flow measurement are provided. Individual analytes are detected in the through-flow, and a dynamic detection of a changing analyte state is carried out, for example with respect to the analyte size or morphology. For this purpose, the analytes to be detected, such as cells for example, are directly marked in the medium surrounding said analytes with magnetic labels and transported through the flow channel of a measuring device comprising at least two magnetic sensors. A characteristic measurement signal is generated by means of the magnetic sensors that are mutually spaced in the flow direction. The magnetic analyte diameter is calculated using the interval between the measurement deflections, and the analyte state can be evaluated using the magnetic analyte diameter.

### CROSS-REFERENCE TO RELATED APPLICATIONS

The present patent document is a §371 nationalization of PCT Application Ser. No. PCT/EP2012/064991, filed Aug. 1, 2012, designating the United States, which is hereby incorporated by reference, and this patent document also claims the benefit of DE 10 2011 080 945.7, filed on Aug. 15, 2011, which is also hereby incorporated by reference.

### TECHNICAL FIELD

The present embodiments relate to the technical field of magnetic flow measurement of magnetically marked analytes, and in particular, to magnetic flow cytometry.

### BACKGROUND

In the field of analyte measurement, and in particular, cell measurement, microscopy methods and scattered light methods are known for determination of the analyte size and shape, or morphology. By scattered light measurements, for instance, in optical flow cytometry, the cell morphology and the cell diameter are detected by so-called forward or side scattering, or a combination of the two. However, scattered light measurements require the sample preparation that may lead to cell stress, which may thus already modify or destroy the cell during the sample preparation. Therefore, in particular, dynamic changes in the quantity to be determined, such as diameter or morphology that take place within a few minutes, are not accessible with this method.

By microscopic methods, in turn, cell concentration determination may not be carried out, or may be carried out only with great difficulty. Simultaneous acquisition of the cell concentration and a dynamic change in the cell shape that gives an indication of the cell state is therefore not possible with previously known methods. Especially in the field of analysis in diagnosis and in the so-called life sciences, however, it is important to selectively detect cells from a complex suspension, for instance a blood sample, and to establish the dynamic change thereof.

### SUMMARY AND DESCRIPTION

It is an object of the present embodiments to provide a suitable method for dynamic acquisition of analyte changes over time, where the concentration of the analyte in a sample may also be determined.

In the method according to an embodiment for magnetic flow measurement of an analyte, the following acts are carried out: magnetic marking of analytes in a sample, generation of a gradient magnetic field at least in the acquisition region of a sensor arrangement, and flow generation of the analytes over the sensor arrangement, the flow of the analytes being guided initially over a first magnetoresistive component and subsequently over a second magnetoresistive component. Detection of individual marked analytes, in which at least three measurement excursions are recorded per analyte, is carried out. The measurement excursions are induced by the stray magnetic field of each marked analyte. The at least three recorded measurement excursions form a characteristic measurement signal for single-analyte detection. Subsequently, evaluation of the measurement signal, in which the measurement signal is identified as single-analyte detection with the aid of the measurement excursion sequence, and in which the analyte magnetic diameter is calculated with the aid of the measurement excursion separation, is carried out. The analyte state is evaluated with the aid of the analyte magnetic diameter.

The method thus uses, in particular, the fact that the magnetic diameter, established by the stray field maximum of the marked analyte, is less than the hydrodynamic or optical diameter, as is determined, for example, with the Coulter counter method. This means that the majority of the stray magnetic field of the immunomagnetic marking extends through the interior of the cell. The analytes are, in particular, cells and may be present in blood samples. By distinguishing the analyte optical diameter from the analyte magnetic diameter, the analyte state may be evaluated since, with the same marking, the magnetic diameter varies with the state.

The method is used to detect analytes that may assume at least a first state and a second state, the two states being manifested by a change in the analyte magnetic diameter. The state change of the analytes does not simultaneously lead to a change in the analyte hydrodynamic diameter.

Discrimination between rounded and confluent test cells, for instance, has to date been possible only by elaborate microscopy. By recording the cell magnetic diameter, it is now possible to carry out this discrimination even in a flow measurement.

Another example is the change of a thrombocyte as a result of its so-called activation. If the thrombocyte when inactive is in the form of a flattened ellipsoid or platelet, the magnetic diameter lies inside the cell. In the activated state, however, when the thrombocyte includes a multiplicity of projections on the surface, the so-called pseudopodia, the cell's magnetic diameter changes such that the magnetic diameter actually lies outside the hydrodynamic diameter and the magnetic diameter exceeds the hydrodynamic diameter by up to 30%. The method thus has the substantial advantage of being able to discriminate between activated and nonactivated thrombocytes, and at the same time of being able to determine their concentration, for example, in a stabilized whole blood sample. The method thus offers an advantageous possibility for thrombocyte function diagnosis.

Thus, in the method, for example, a state change that is a change in the cell morphology is evaluated, and/or a state change that is attributable to a change in the cell geometry is evaluated, as described in the examples mentioned above.

In an advantageous embodiment, a plurality of subsequent measurements are carried out in the method in order to detect individual analytes, so that a dynamic change in the analyte state is recorded with the aid of its magnetic diameter. The method thus has the advantage of not only detecting an analyte state at a single time but of tracking the analyte state over a period of time. The immunomagnetic marking of the analytes, which may be cells, is used in the method so that a dynamic change in the cell state over a time range of from one second to one hour may be observed. Furthermore, the method has the advantage of also determining the cell concentration as a function of time.

For example, the plurality of successive measurements is carried out by guiding the flow of the analytes over a plurality of sensor arrangements, each of the plurality of sensor arrangements having a first magnetoresistive component and a second magnetoresistive component. Alternatively, the sample is guided several times in succession over one or more sensor arrangements.

The method thus offers the advantage not only of being able to detect an analyte state just once, but of dynamically recording and evaluating its variation over time. This may also be contributed to by the magnetic flow measurement, since in this way, other than the addition of magnetic markers, scarcely any sample preparation that would stress or even destroy the cells has to be carried out. Marked cells may be observed in the second to hour range with the described method.

In the method, advantageously, the magnetic marking is carried out with magnetic nanobeads, e.g., superparamagnetic nanobeads. Nanobeads have, for example, hydrodynamic diameters of between 10 nm and 500 nm. Magnetic nanobeads that have magnetite or maghemite as material are advantageous. The marking is carried out, for example, such that the occupation density of the markers on the analyte surface is between 10% and 90%, for example, depending on the cell surface and the epitope number on the cell surface. The material magnetite, for example, has a saturation magnetization of about 80 to 90 (A·m²)/kg. Depending on the proportion of the material, or if another material is contained in the nanobeads, the superparamagnetic labels have a saturation magnetization of between about 10 and 60 (A·m²)/kg. Such magnetic marking is advantageous since the stray magnetic field, caused by the marking in the gradient magnetic field, extends predominantly through the interior of the cell, and in particular the maxima of the stray field lie inside the cell diameter. In this way, the method additionally allows that even analytes directly following another analyte, which move over the sensor arrangement, may be read individually. In the case of a stray magnetic field that for the most part lies outside the cell, a signal overlap of the magnetoresistive signal occurs that no longer allows unambiguity of single-analyte detection.

In another advantageous embodiment, the flow speed of the sample in the method is adjusted such that the analytes are guided over the magnetoresistive components with a constant speed. For example, by adjustment of the flow speed, the analytes are caused to roll over the magnetoresistive components.

In another advantageous embodiment, a preparation act is carried out in the method where the channel inner wall is modified such that the state of the analytes to be detected is altered upon contact with the channel inner wall. In particular, this state change may be activation of cell analytes. Thrombocytes are one example of this. If thrombocytes are being detected by the described method, the thrombocytes may be activated upon contact with a suitably prepared channel inner wall that leads to a change in the cell morphology. As already described, the thrombocyte changes from a flat ellipsoid to a structure having very many projections from the surface. Such a state change is manifested clearly in the magnetic radius and may be detected by the described method.

Statistical distributions of diameters may be recorded by the described method. Even in the case of only one analyte type to be detected being present, the analytes have different hydrodynamic diameters and different magnetic diameters.

The device according to an embodiment for flow measurement includes a flow channel, at least one magnetic unit that is arranged below the channel bottom of the flow channel and is configured in order to generate a gradient magnetic field that permeates the volume enclosed by the flow channel, at least one cell measurement instrument having at least two magnetoresistive components, the magnetic unit being configured in order to induce a gradient field having a magnetic field strength of between 1 mT and 500 mT, each magnetoresistive component being arranged at a distance of between 0.2 μm and 40 μm from another magnetoresistive component, and the channel inner wall being configured such that the adhesion force of the analytes with the surface of the channel inner wall is less than the shear force for flow speeds of between 0.1 mm/s and 10 mm/s.

In an advantageous embodiment of the device, the flow channel is configured in respect of channel diameter and surface condition of the channel inner wall such that, with a predetermined gradient magnetic field and predetermined flow speed, shear forces acting on the analytes are less than 1 g/(cm·s²) and/or the Reynolds number is less than 2000.

Advantageously, each magnetoresistive component is arranged at a maximum distance from another magnetoresistive component so that the analytes pass over the cell measurement instrument essentially with a constant speed. The maximum separation between magnetoresistive components is, for example, less than 40 μm, or less than 30 μm. In this way, a sufficient signal amplitude with a constant external magnetic field is possible.

In certain embodiments, the magnetoresistive components are giant magnetoresistance (GMR) sensors. The magnetoresistive components are, in particular, arranged in a Wheatstone bridge circuit. At least two magnetoresistors may be interconnected as a half-bridge. The advantageous use of a Wheatstone bridge is known, for example, from the patent application DE 10 2010 040 391.1.

In particular, the described device may also have a magnetophoretic enrichment path, as is known for example from the patent application DE 10 2009 047 801.9.

The scope of the present invention is defined solely by the appended claims and is not affected to any degree by the statements within this summary. The present embodiments may obviate one or more of the drawbacks or limitations in the related art.

### BRIEF DESCRIPTION OF THE DRAWINGS

FIG. 1 depicts a side view of one embodiment of a sensor arrangement.

FIG. 2 depicts a diagram of one embodiment of a measurement signal.

FIG. 3 depicts a diagram of one embodiment of an analyte distribution.

FIG. 4 depicts a schematic representation of one embodiment of inactive thrombocytes.

FIG. 5 depicts a schematic representation of one embodiment of activated thrombocytes.

FIG. 6 depicts a side view of one embodiment of a flow channel with inactive analytes.

FIG. 7 depicts a similar view as FIG. 6 of one embodiment of a flow channel with activated analytes.

FIG. 8 depicts a similar view as FIGS. 6 and 7 of one embodiment of a flow channel, the stray magnetic fields of the analytes being shown.

### DETAILED DESCRIPTION

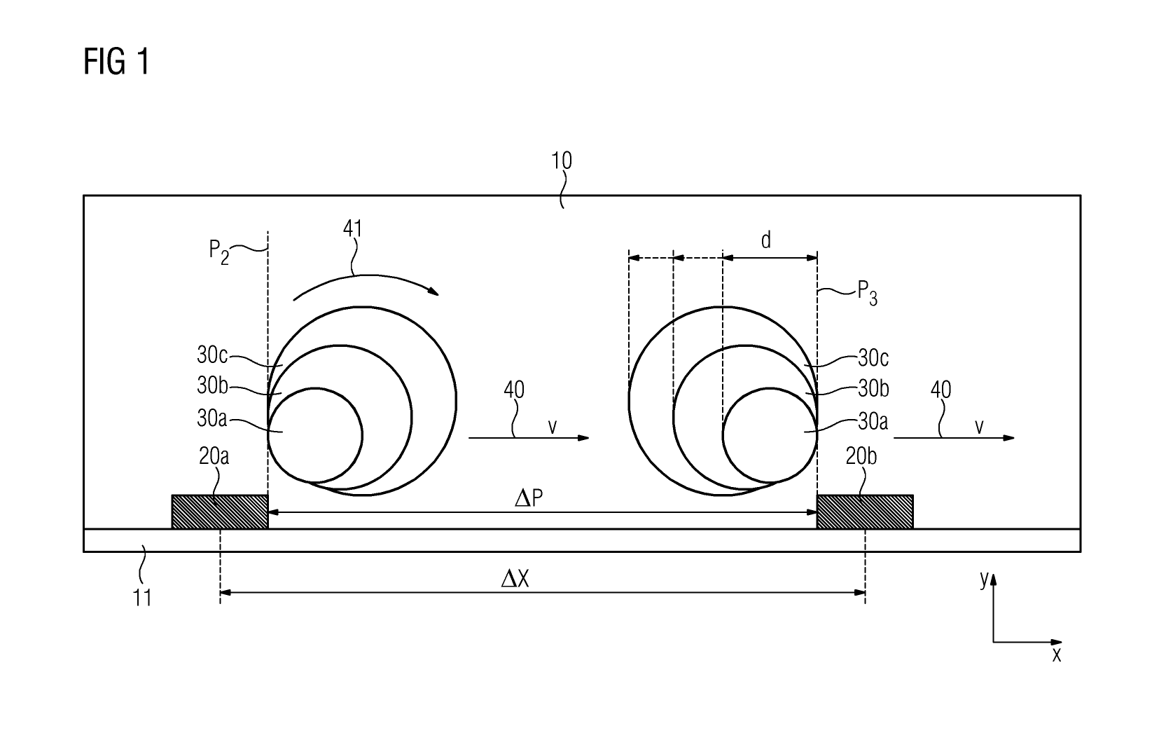

FIG. 1 shows a detail of a flow channel 10 that leads from left to right in the figure. The channel bottom 11 of the flow channel 10 carries two magnetic sensors 20a, 20b, represented in a very simplified way as rectangles in the side view that are flowed over successively in the flow direction 40. The flow direction 40 is indicated by two velocity arrows v from left to right in the flow channel. The two sensor elements 20a, 20b lie at a center distance Δx from each other that may, for example, be adapted to the analyte type to be detected and, in particular, to the analyte diameter d. Analytes 30a, 30b, 30c that move in the flow direction 40, are also represented as circles of different diameter d in the flow channel 10. A curved arrow 41 indicates that the analytes 30a, 30b, 30c rotate while moving forward in the flow 40. In particular, the analytes 30a, 30b, 30c roll over the channel bottom 11.

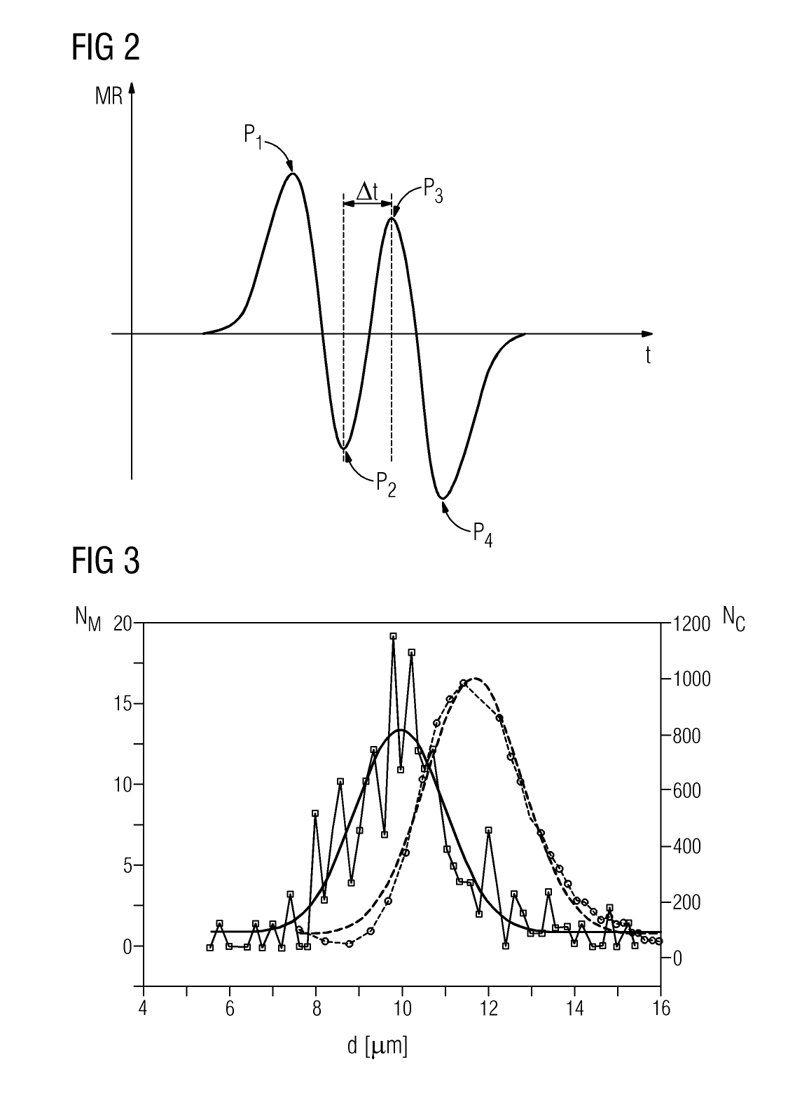

The sensor elements 20a, 20b are in particular magnetoresistive components, that is to say the sensor elements 20a, 20b are in particular magnetoresistors that are connected to one another in a Wheatstone bridge circuit. When two sensors are used, the sensors form in particular a half-bridge. An advantage of the interconnection is the magnetoresistive signal MR that may thereby be generated, represented in a signal/time diagram in FIG. 2.

In the time profile of the passage of an analyte 30a, 30b, 30c over the two sensor elements 20a, 20b, a peak sequence of at least three, or for example even four peaks P1, P2, P3, P4, as shown in FIG. 2, is generated. In this case, the first peak P1 occurs when the analyte 30a, 30b, 30c just reaches the first sensor 20a, and the second peak P2 occurs when the analyte 30a, 30b, 30c has just passed over the first sensor 20a.

This position is also indicated in FIG. 1 by a dashed line P2 extending perpendicularly to the channel bottom 11. The third peak P3 in turn occurs when the analyte 30a, 30b, 30c just reaches the second sensor 20b, as is likewise shown in FIG. 1 again by a dashed line P3 extending perpendicularly to the channel bottom 11. Lastly, the fourth excursion P4 occurs when the analyte 30a, 30b, 30c has also passed fully over the second sensor 20b.

That is to say, the time separation Δt between the second peak P2 and the third peak P3, as indicated in the diagram in FIG. 2, is correlated with the distance ΔP between the two sensors 20a, 20b by the flow speed v. Furthermore, this peak separation Δt also depends on how large the analyte 30a, 30b, 30c is. The larger the analyte diameter d is, the faster the analyte 30a, 30b, 30c is transported in the flow channel 10, and the shorter is the time Δt that elapses between the second and third peaks, or the associated positions P2, P3. By the characteristic measurement signal MR with four measurement excursions P1-P4, single-analyte detection is also possible. The flow speed v may, for example, be determined from the time separation of the first peak P1 and the third peak P3.

Lastly, FIG. 3 shows a further diagram that shows the distribution of the analytes around their measured diameter d. The diameter d is indicated in micrometers. The cell distribution N_{M} is plotted on the left-hand axis as a function of the magnetic cell diameter d measured as described above, and the cell distribution N_{C} that is based on the optical or hydrodynamic diameter of the analyte, as determined in particular by the Coulter method, is plotted on the right-hand axis of the diagram. Here, it is immediately clear that the two diameters, determined by the magnetic measurement or by the Coulter measurement, do not match. The magnetic diameter is in this case, for example, about 2 μm less than the Coulter diameter. This shift is attributable to the fact that, when passing over the sensor units 20a, 20b, it is not the actual analyte or cell diameter d that is responsible for the measurement excursions P1-P4, but the edge 26 of the stray magnetic field 24, or its maximum 26 of the x component of the stray field 24 that is recorded by the sensors 20a, 20b.

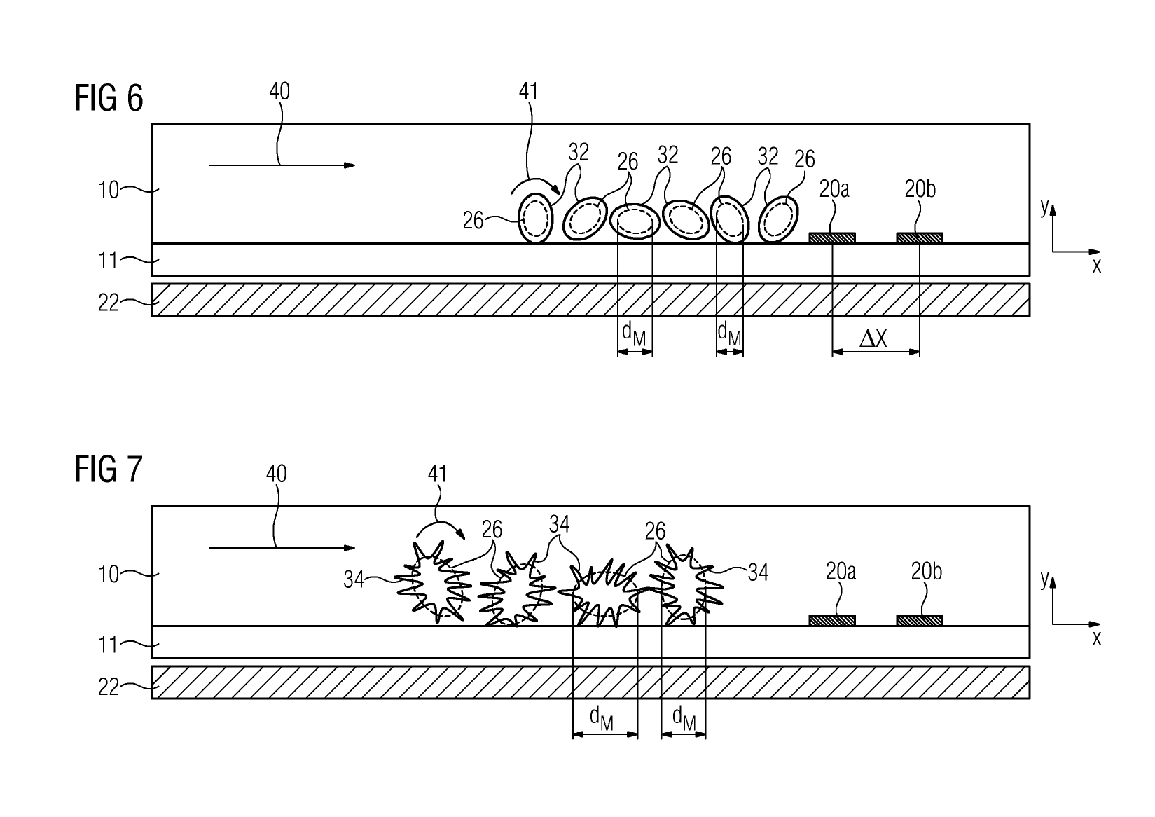

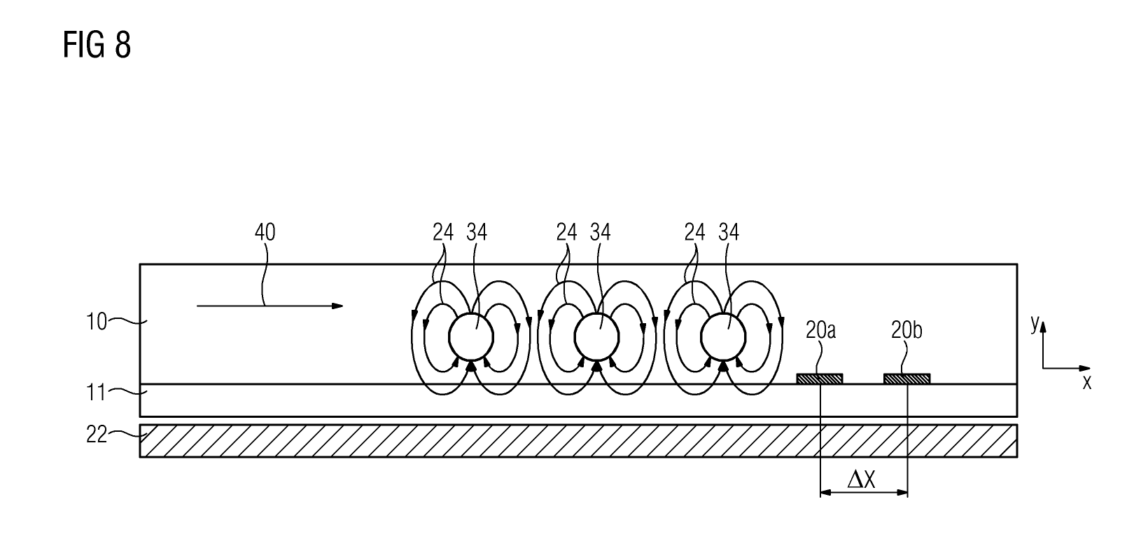

Although the magnetic marking is applied externally on the analyte surface, the stray magnetic field maximum may also lie inside the cell 32, 34. The magnetic field lines 24 of the magnetically marked cells 32, 34 are, for example, shown in FIG. 8. The differently lying magnetic diameters d_{M} are shown in FIGS. 6 and 7. In this case, it is not only the magnetic marking, but in particular also the shape and surface of the analyte 32, 34 that play a role as to whether the magnetic diameter d_{M} lies inside or outside the analyte 32, 34 to be detected.

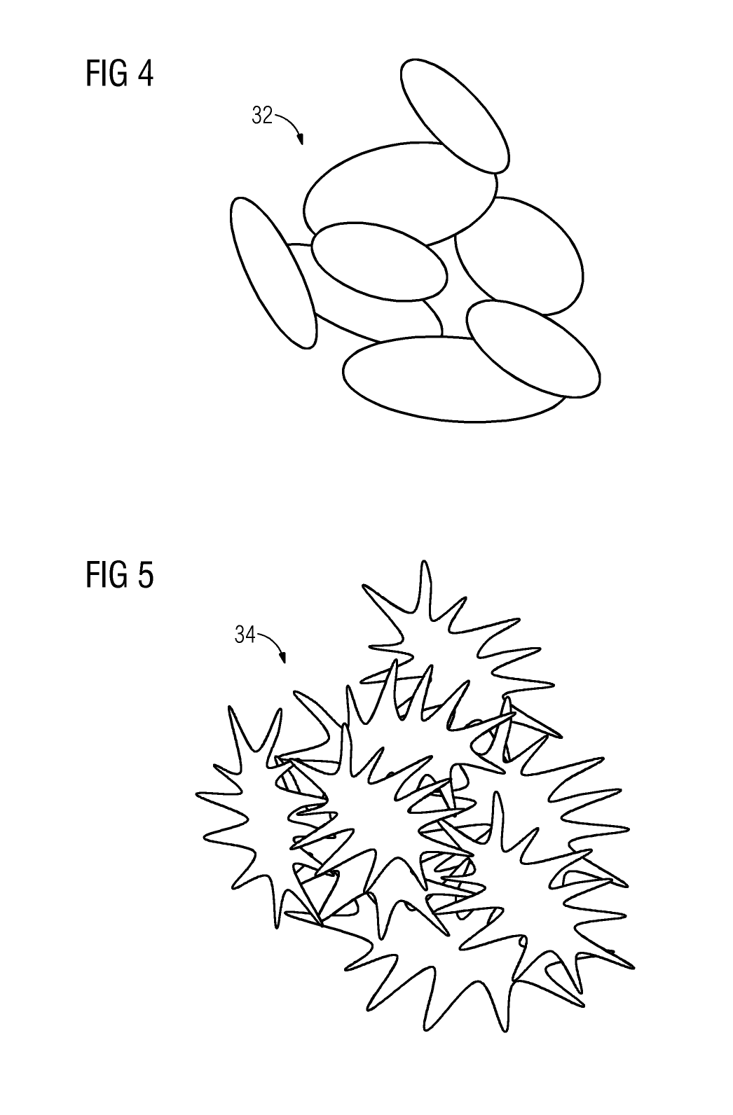

FIGS. 4 and 5 show very simplified schematic representations of cells 32, 34 that may in particular constitute thrombocytes. In a first state 32, these are compact ellipsoids as represented in FIG. 4, and in a second state 34 the thrombocytes are very irregularly shaped and have a multiplicity of extending projections, so-called pseudopodia. These represent a very great surface enlargement of the cell 34.

As shown in the subsequent FIGS. 6 and 7, for the same cell and the same magnetic marking, the diameter d_{M} of the stray magnetic field is altered by the state change from inactive platelets 32 to activated thrombocytes 34. In the case of the compact ellipsoids 32, the magnetic diameter d_{M} lies inside the cell, while in the case of the activated cell 34 with the projecting pseudopodia, the maximum 26 of the stray magnetic field 24 moves outward, so that the magnetic diameter d_{M} becomes greater than the actual cell diameter d.

FIGS. 6 to 8 again show the flow channel 10 with the channel bottom 11 and the sensor arrangement 20a, 20b as in FIG. 1. The magnetic unit 22, a permanent magnet, is arranged below the channel bottom 11. In the surface extent of the magnetic unit 22, the magnetic unit 22 is in particular large enough so that a homogeneous gradient magnetic field that enriches the magnetically marked cells 32, 34 on the channel bottom 11, may be generated throughout the entire volume of the flow channel 10.

Lastly, FIG. 8 once more shows the cross section through the flow channel 10 in a similar way to FIGS. 6 and 7. Magnetically marked cells 32, 34 and the magnetic field lines 24 of the stray magnetic field, generated by the magnetic marking of the cells 32, 34, are indicated. Of this stray field 24, the sensors 24a, 24b detect in particular the x component. The x direction is indicated by the diagram by the side of the flow channel 10, and points in the direction of the flow 40.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for magnetic flow measurement of an analyte, the method comprising:
magnetically marking analytes in a sample;
generating a gradient magnetic field at least in an acquisition region of a sensor arrangement;
guiding the analytes over the sensor arrangement in a flow direction, the flow direction of the analytes being initially over a first magnetoresistive component and subsequently over a second magnetoresistive component;
detecting the analytes by recording, per analyte, at least three measurement excursions induced by a stray magnetic field of each of the analytes, thereby forming a characteristic measurement signal for single-analyte detection;
evaluating the characteristic measurement signal, wherein the characteristic measurement signal is identified as the single-analyte detection with aid of a measurement excursion sequence, and wherein an analyte magnetic diameter is calculated with aid of a measurement excursion separation; and
evaluating an analyte state with aid of the analyte magnetic diameter.

2. The method as claimed in claim 1, wherein an analyte to be detected changes from a first state to a second state, defining a state change, and manifested by a change in the analyte magnetic diameter.

3. The method as claimed in claim 2, wherein the state change is not simultaneously manifested by a change in an analyte hydrodynamic diameter.

4. The method as claimed in claim 2, wherein the state change is a change in cell morphology.

5. The method as claimed in claim 2, wherein the state change is a change in cell geometry.

6. The method as claimed in claim 1, wherein the detection of each of the analytes is carried out in a plurality of successive measurements, so that a dynamic change in the analyte state is recorded with the aid of a magnetic diameter.

7. The method as claimed in claim 6, wherein the plurality of successive measurements is carried out by guiding the flow of the analytes over a plurality of sensor arrangements, each of the plurality of sensor arrangements having two magnetoresistive components.

8. The method as claimed in claim 1, wherein the magnetically marking is carried out with magnetic nanobeads.

9. The method as claimed in claim 1, wherein a flow speed of the sample is adjusted such that the analytes pass over the first magnetoresistive component and the second magnetoresistive component.

10. The method as claimed in claim 1, wherein the guiding of the flow is through a flow channel having a channel inner wall, the method further comprising:
modifying the channel inner wall such that the analytes to be detected are altered upon contact with the channel inner wall.

11. The method as claimed in claim 10, wherein the analytes to be detected are cells and the state change is activation of the cells.

12. The method as claimed in claim 10, wherein the analytes to be detected are thrombocytes and the state change is activation of the thrombocytes.

13. A device for flow measurement, the device comprising:
a flow channel having a channel inner wall,
a magnetic unit arranged below a channel bottom of the flow channel and configured in order to generate a gradient magnetic field that permeates the volume enclosed by the flow channel,
at least one cell measurement instrument having a first magnetoresistive components and a second magnetoresistive component,
wherein the magnetic unit is configured in order to induce a gradient field having a magnetic field strength of between 1 mT and 500 mT,
wherein the first magnetoresistive component is arranged at a distance of between 0.2 μm and 40 μm from the second magnetoresistive component, and
wherein the channel inner wall is configured such that the adhesion forces of analytes with the surface of the channel inner wall is less than the shear forces of the analytes for flow speeds of between 0.1 mm/s and 10 mm/s.

14. The device as claimed in claim 13, wherein the flow channel is configured in respect of a channel diameter and a surface condition of the channel inner wall such that, with a predetermined gradient magnetic field and a predetermined flow speed;
the shear forces acting on the analytes are less than 1 g/(cm·s²), the Reynolds number is less than 2000, or the shear forces are less than 1 g/(cm·s²) and the Reynolds number is less than 2000.

15. The device as claimed in claim 13, wherein the first magnetoresistive component is arranged at a maximum distance from the second magnetoresistive component so that the analytes pass over the cell measurement instrument essentially with a constant speed.

16. The device as claimed in claim 14, wherein the first magnetoresistive component is arranged at a maximum distance from the second magnetoresistive component so that the analytes pass over the cell measurement instrument essentially with a constant speed

17. The method as claimed in claim 11, wherein the analytes to be detected are thrombocytes and the state change is activation of the thrombocytes.

18. The method as claimed in claim 8, wherein a flow speed of the sample is adjusted such that the analytes pass over the first magnetoresistive component and the second magnetoresistive component.

19. The method as claimed claim 9, wherein the guiding of the flow is through a flow channel having a channel inner wall, the method further comprising:
modifying the channel inner wall such that the analytes to be detected is are altered upon contact with the channel inner wall.
